(19) United States
(12) Patent Application Publication
(10) Pub. No.: US 2005/0043630 A1
(43) Pub. Date: Feb. 24, 2005

(54) Thermal Emission Non-Invasive Analyte Monitor

(76) Inventor: Janusz Michal Buchert, New York, NY (US)

Correspondence Address: Janusz M. Buchert, 180 Cabrini Blvd., #79, New York, NY, 10033, US

(21) Appl. No.: 10/604,851

(22) Filed: Aug. 21, 2003

(51) Int. Cl. A61B 6/00

(52) U.S. Cl. 600/473

(57) ABSTRACT

An improved method and an improved instrument for analyte determination that uses infrared radiation naturally emitted by subject are disclosed. The method is based on Thermal Emission Spectroscopy (TES) whereby the spectral signal is measured in reference to a body's physiological and ambient parameters. The instrument that realizes the method incorporates temperature and humidity sensors. Ambient environmental parameters and subject parameters as disclosed allow normalization of spectrally specific analyte signal for grater precision and accuracy of analytes concentration determination. Such improvement leads to a universal calibration in, for example, non-invasive blood glucose measurements in human subjects.

### BACKGROUND OF INVENTION

1. Field of the Invention

The present invention relates generally to an infrared spectral measurement method and instrument that uses infrared radiation naturally emitted by a subject in mid- and far-infrared spectral regions and is based on Thermal Emission Spectroscopy (TES) analytical method for non-invasive determination of analytes concentration. It relates more specifically to the method and instrument that incorporates ambient temperature and humidity sensors as well as physiological temperature sensors. Said method and said instrument allow a better normalization of spectrally specific analyte signal for greater precision and accuracy of analytes concentration determination. It leads to universal calibration in, for example, non-invasive blood glucose measurements in human subjects.

2. Related Art

At least 170 million people suffer from diabetes world-wide and two thirds of them live in developing countries, according to the World Health Organization (WHO). The number of newly diagnosed people with diabetes is increasing at all ages, and notably in younger age groups. In many developing countries, the prevalence of diabetes in adults is now greater than 10%. Most of the health impact of diabetes is the result of its long-term complications and eye problems retinopathy and cataracts are among the most distressing and costly to society. Sixteen million people in the United States live with the chronic disease diabetes; approximately 5-10% are children. The seminal Diabetes Control & Complications Trial (The Diabetes Control and Complications Trial Research Group, New Engl. J. Med. 329:977-1036, 1993) concluded that frequent glucose monitoring is necessary to reduce the complications of this disease. A lack of compliance occurs despite strong evidence that tight control dramatically reduces long-term diabetic complications. However, all glucose monitors available require invasive techniques with the most widely used method of self-monitoring, obtaining blood from a finger prick, causing pain and discomfort which results in poor compliance. A novel, hand-held, non-invasive glucose monitor will provide diabetics with the means for testing their glucose level more frequently, improving their quality of life, and reducing the costs and complications of this chronic disease.

The present invention is an improvement of a method and an improvement of an instrument based on the prior art discovery (U.S. Pat. No. 5,666,956 and U.S. Pat. No. 5,823,966 issued to J. M. Buchert, the entire contents of these patents are hereby incorporated herein by reference and made a part of this specification) that natural infrared thermal emission from tissue or organs of the subject, which could be any mammal species, is modulated by the state of the emitting source. The thermal infrared radiation from any matter at temperature above zero degrees Kelvin consists of spectral information defining the state of the emitting matter. Spectral information comprised in said emission consists of spectral information of the subject tissue. By measuring thermal emission spectral features of certain analytes the concentrations of analytes can be determined in a non-invasive manner.

Thermal emission was first applied medically in 1957 when Lawson (Lawson R: “Implication of Surface Temperatures in the Diagnosis of Breast Cancer”, Can Med Assoc J: 75:309-310, 1956) discovered that skin temperature over a cancer in the breast was higher than that of normal tissue. Thermal imaging is a noninvasive diagnostic technique that allows the examiner to visualize and quantify changes in skin surface temperature. Thermography's major clinical value is its high sensitivity to pathology in the vascular, muscular, neural and skeletal systems and as such it can contribute to the pathogenesis and diagnosis made by the clinician. It has been used extensively in human medicine in the U.S.A., Europe and Asia for the past 20 years.

In 1987 Jacob Fraden patented (U.S. Pat. No. 4,797,840) an instant ear thermometer that measured the intensity of infrared radiation emitted from the tympanic membrane (eardrum). Tympanic membrane thermometers currently widely used at home and in the hospital environment determines temperature by utilizing total energy from a wide spectral range of the human body heat infrared emission, which is usually contained between 8 and 14 micrometers.

Prior art (U.S. Pat. No. 5,666,956 and U.S. Pat. No. 5,823,966) and present invention takes much further the technology based on Thermal Emission Spectroscopy (TES), using a spectral analysis of the infrared thermal emissions to measure tissue analyte's concentration. This new cost-effective, painless blood glucose monitor will improve patient compliance and should thereby reduce diabetic complications and their high cost.

This technology can also be adapted for use as a continuous monitor (U.S. Pat. No. 5,823,966) and, with various control algorithms, could provide a closed-loop feedback system with insulin delivery devices.

Blood glucose concentrations may be measured by invasive or minimally invasive techniques. Some of these methods measure blood glucose directly and some measure interstitial fluid glucose. For example, Cygnus, Inc.'s GlucoWatch (“GlucoWatch Automatic Glucose Biographer and Autosensor”: available from http://www.glucowatch.com/us/prescribing_info/prescribing_info.pdf) is the only minimally invasive instrument approved by FDA as an adjunctive device to supplement blood glucose testing. The device transdermally extracts interstitial fluid from the skin using iontophoresis. An extremely low electric current pulls interstitial fluid glucose through the skin. However, the GlucoWatch still requires daily calibration of the instrument using the invasive finger-stick method. MiniMed Inc.'s product (“Medtronic/MiniMed CGMS specifications”, available from http://www.minimed.com/doctors/md_products_cgms_specs.shtml) is a subcutaneous, continuous blood glucose monitoring system that directly records and stores concentration values in memory. This invasive device does not provide measurements directly to the patient and is available for professional use only.

There are a number of reviews (Klonoff D C, “Non-invasive Blood Glucose Monitoring”, Diabetes Care 20:433-437, 1997; Koshinsky T, Heinemann L, “Sensors for Glucose Monitoring: Technical and Clinical Aspects”, Diabetes Metab Res Rev 17: 113-123, 2001) on approaches for non-invasive blood glucose measurements. In recent years, infrared (IR) spectroscopy has emerged as the analytical method of choice founded on the spectrum of IR frequencies characteristic of the analyte itself instead of relying on reagents and color reactions. Kaiser (U.S. Pat. No. 4,169,676) showed the possibility of a non-invasive method of glucose measurement by analyzing the infrared absorption spectrum through an attenuated total reflection (ATR) prism. Others (Kajiwara et al. “Spectroscopic Quantitative Analysis of Blood Glucose by Fourier Transform Infrared Spectroscopy with an Attenuated Total Reflection Prism”, Med Prog Technol 18: 181-189, 1992) reported using Fourier Transformed Infrared spectroscopy (FTIR) methods for quantitative measurements of glucose concentration in blood and serum samples at characteristic absorbance peaks. Different approaches in infrared absorption are described in following references: Bauer et al., “Monitoring of Glucose in Biological Fluids by Fourier-Transform Infrared Spectrometry with a Cylindrical Internal Reflectance Cell”, Analytica Chimica Acta 197: 295-301, 1987; Bhandare et a., “Glucose Determination in Simulated Plasma Blood Serum Solutions by FTIR Spectroscopy: Investigation of Spectral Interferences”, Vibrational Spectroscopy 6: 363-378, 1994; Heise et al., “Multi-component Assay for Blood Substrates in Human Plasma by Mid-infrared Spectroscopy and its Evaluation for Clinical Analysis”, Applied Spectroscopy, 48: 85-95, 1994; Cadet F, “Method for the Classification of Biological FT-IR Spectra Prior to Quantitative Analysis”, Applied Spectroscopy 50: 1590-1596, 1996; Budinova et al., “Application of Molecular Spectroscopy in the Mid-infrared Region to the Determination of Glucose and Cholesterol in Whole Blood and in Blood Serum”, Applied Spectroscopy 51:631-635, 1997; Vonach et al., “Application of Mid-Infrared Transmission Spectrometry to the Direct Determination of Glucose in Whole Blood”, Applied Spectroscopy 52: 820-822, 1998. None of these devices are commercially available. These devices utilize absorption, transmission, and reflection methods in order to spectroscopically analyze blood glucose concentration. Near-infrared (NIR) spectroscopy techniques, developed by companies such as Instrumentation Metrics (Sensys) and LifeTrac, are fundamentally different from thermal emission midinfrared methodology. These methods require an outside near-infrared (NIR) excitation source to measure the resulting radiation after interaction with the tissue. The near-infrared spectrum is not very selective for blood glucose determination because it relies on the overtone and combinational absorption and not on the fundamental spectral fingerprint of glucose in the mid-infrared region.

Argose Inc. is trying to develop another optical technology based on UV-induced fluorescence where the target is not glucose itself, but a molecular component of skin (e.g. tryptophan or collagen cross-links) that fluoresces in relation to its glucose concentration. This indirect spectral method is poorly correlated with blood glucose and the chronic use of UV light could be harmful to human tissue.

Knudson in U.S. Pat. Nos. 5,115,133; 5,146,091 and 5,179,951 discloses a method for measuring blood sugar that involves testing body fluid constituents by measuring light reflected from the tympanic membrane. A testing light and a reference light at a glucose sensitive wavelength at about 500 to about 4000 wave numbers (cm⁻¹) are directed toward the tympanic membrane which contains fluid having an unknown concentration of a constituent. A light detector is provided for measuring the intensity of the testing light and the intensity of the reference light, both of which are reflected and spectrally modified by the fluid. A light path distance measurer is provided for measuring the distance of a light path traveled by the testing light and the reference light. A circuit is provided for calculating the level of the constituent in the fluid in response to a reduction in intensity in both the testing light and the reference light and in response to the measured distance. Knudson teaches that measurements of a body fluid constituent can be performed by measuring across the tympanic membrane and using the absorption method that is characterized by light generating means for generating a testing light of known intensity, with said testing light including at least one wavelength absorbable by said constituents and further determining the amount of said testing light absorbed by said constituent.

Optiscan's Biomedical Corporation (U.S. Pat. Nos. 5,515,847 and 5,615,672) technology relies on monitoring the infrared absorption signal through the wrist. Measurements are made by monitoring infrared absorption of the desired blood constituent in the long infrared wave-length range. It uses human body heat radiation as a source radiation for measurements of resulting transmission through arterial blood in the wrist. It consists of an infrared detector, which detects light at infrared wave-lengths that has passed through the arterial blood vessel of the patient and has been selectively absorbed by at least one predetermined constituent at characteristic infrared absorption wavelengths for these constituent. Unfortunately, the thick skin of the wrist is not penetrable to infrared radiation, so the skin must be cooled down and then warmed up (Optiscan issued U.S. Pat. Nos.: 55,900,632; 6,025,597; 6,049,081; 6,072,180; 6,161,028; 6,198,949; 6,556,850; 6,577,885; 6,580,934) to obtain glucose spectral information from the dermis's vasculature. Optiscan device relies on the capture of thermal gradient spectra from living tissue by periodic temperature modulation and phase detection. The instrument has a size of a desktop computer and consists of mechanical parts of a complicated design, limiting its portability. Portability could be also limited by the size of the energy source (a battery) required for frequent warming up and cooling down of the tissue.

In the U.S. Pat. No. 6,002,953 issued to Block, a noninvasive infrared transmission measurement of analyte in the tympanic membrane is disclosed. The invention cools a segment of the subject's tympanic membrane and employs the thermal radiation emitted by the subject's inner ear and that is transmitted through this cold segment in order to directly obtain absorption information related to the concentration of various constituents of the blood flowing through the membrane. In particular the invention utilizes optical devices inserted into the external ear cavity in order to direct a portion of the transmitted radiation onto an infrared detection and analysis device. The signal from the detection device is analyzed in order to obtain the concentration of the constituent of interest. The invention is similar in approach to Optiscan method of cooling the measured tissue in order to obtain analyte concentration information using absorption spectrum analysis. It is not practical; it would require substantial cooling (as one can find from Optiscan patents) of the ear canal that could be uncomfortable to the user. The dynamics of physical phenomena during the process of tissue cooling additionally complicate the analysis and influence uncertainty of the results.

The invented technology relates to a unique analytical method based on Thermal Emission Spectroscopy (TES reference: Willis H A, “Laboratory Methods in Vibrational Spectroscopy”, New York, J. Willey & Sons, 1987; Chase D B, “The Sensitivity and Limitation of Condensed Phase Infrared Emission Spectroscopy”, Applied Spectroscopy, 35:77-81, 1981; DeBlase et al., F J, “Infrared Emission Spectroscopy: a Theoretical and Experimental Review”, Applied Spectroscopy, 45: 611-618, 1991; Sullivan et al., “Surface Analysis with FT-IR Emission Spectroscopy”, Applied Spectroscopy 46: 811-818, 1992; Keresztury et al., “Quantitative Aspects of FT-IR Emission Spectroscopy and Simulation of Emission-Absorption Spectra”, Analytical Chemistry 67: 3782-3787, 1995; Friedrich et al., “Emission Spectroscopy: An Excellent Tool for the Infrared Characterization of Textile Fibers”, Applied Spectroscopy 52:1530-1535, 1998), which was used, for example, during the Mars expedition (by NASA) to analyze the chemistry of Martian rocks and is being used in astronomy to analyze chemical components of stars.

The various spectroscopic methods and instruments that aim to monitor sample temperature for spectral noninvasive blood glucose monitoring are described in the prior art.

For example Braig et al. in U.S. Pat. No. 5,615,672 describe a glucose monitor that non-invasively measures glucose concentration by performing the absorption analysis based on body heat infrared radiation and its transmission through arterial blood in the wrist. The described device includes a temperature-sensing device for measuring the person's internal temperature at the arm to adjust the constituent concentration measurement for temperature-dependent effects. However, the sensor measures temperature at the skin surface and is not integrated into the device. Therefore, the calculated compensation for internal body temperature, to be applied to the measured spectral signal, introduces a significant source of error in the analyte concentration estimate.

Another U.S. Patent Application Publication U.S. 2002/0038080 A1 by Makarewicz et al. describes a method and apparatus for minimizing the effects caused by fluctuation in tissue state upon a noninvasive in-vivo near-infrared spectral measurement. Selected tissue state parameters are monitored spectroscopically, which allows maintaining these parameters within a target range. The invention provides a method and apparatus for minimizing effects in near infrared (NIR) spectral measurements variation due to skin temperature changes at a tissue measurement site. It is an especially dominant problem in the near infrared spectral region, as shown by Jensen et al. in the paper “Influence of Temperature on Water and Aqueous Glucose Absorption Spectra in the Near- and Mid-infrared Regions at Physiologically Relevant Temperatures”, Applied Spectroscopy: 57(1) 28-36, 2003.

The ear non-invasive blood glucose monitor (prior art method and instrument described in U.S. Pat. No. 5,666,956 and U.S. Pat. No. 5,823,966) is an infrared spectral monitor, which measures the infrared radiation from the subject tympanic membrane naturally emitted as heat in a manner similar to a non-contact ear tympanic thermometer. While infrared thermometer is measuring total infrared spectrum over a wide range of wavelengths emitted by the tympanic membrane, the infrared glucose monitor distinguishes between different spectral lines to correlate their properties with glucose concentration. In the case of the invented instrument, the spectral signatures (e.g. of glucose) contained in such broadband infrared energy emission from human tissue are used to perform constituent composition and concentration analysis. The device is a very sensitive, portable, hand-held filtometer. The device is passive and does not harm the human tissue by a chronic external radiation.

The use of the invented improved method and the invented improved instrument will prevent instability and uncertainty of proper universal calibration of the noninvasive analyte (e.g. blood glucose) monitor and will allow to reduce the influence of the environmental conditions such as ambient temperature and humidity as well as physiological subject parameters.

### SUMMARY OF INVENTION

In the infrared spectral monitor an appropriate infrared sensor detects the emission spectral lines features, which is an integral part of measurements and/or acquisition system. A signal from the system is usually converted into useful information about analyte concentration in tissues after calibration process performed on real subjects. Calibration process involves monitoring of environmental and physiological subject parameters such as temperature and ambient humidity. It has a purpose of spectrally-specific analyte signal normalization for different subjects, allowing universal calibration. All the above parameters have to be included in non-invasive blood glucose measurements calibration calculation and further for predictive blood glucose measurements. Body temperature of the subjects as a calibration parameter will compensate, for example, for changes of the body thermal emission intensity that are independent of glucose emission intensity changes due to concentration changes. Measurements of these environmental and subjects' parameters and incorporating them into the calibration and prediction algorithm will allow compensation for their influence on the signal that is spectrally-specific to glucose thermal emission in the various subjects and also will allow universal calibration.

If spectral measurements are performed in well-controlled ambient conditions as described for the in vitro experiment in Diabetes Care: 25(12) 2268-2275, 2002, publication, it is not necessary to include additional parameters in calculating of the glucose concentration from the intensities of glucose thermal emission spectral lines. The above is also true for any other laboratory experiment that uses absorption, fluorescence or Raman lines, etc. for the purpose of quantitative substance concentration measurements in vitro. For the measurements in real life conditions, especially in vivo and non-invasive, one must incorporate the necessary environmental and physiological subject's parameters to compensate for their influence on spectral measurements.

Thus, a primary purpose of the present invention is to provide an infrared spectral monitor integrated with temperature and humidity sensors. A further objective is to provide the infrared spectral monitor integrated with ambient temperature sensor. It is still the further objective to provide the subject's body temperature sensor. It is yet still the further objective to provide the subject's tympanic membrane temperature sensor. It is another objective to provide the ambient humidity sensor. It is still another objective of the invention to provide the infrared spectral monitor that is not influenced by environmental conditions such as ambient temperature and humidity. It is yet still another objective of this invention to provide the infrared spectral monitor that is not influenced by the subject's physiological condition, such as body temperature as well as the size and physiological state of the ear canal.

It is still another and further objective of this invention to provide the spectral analyte-specific infrared measurements from the same spot of tissue as the temperature measurements by infrared radiation sensor. It is still another and still further objective of this invention to provide the spectral analyte-specific infrared measurements in a continuous manner.

### BRIEF DESCRIPTION OF DRAWINGS

FIG. 1a is infrared absorption spectrum of D-glucose.

FIG. 1b is infrared absorption spectra of: blood (59 mg/dL), blood added with glucose(371 mg/dL), and a glucose standard solution (1000 mg/dL) in the spectral range of the glucose absorption.

FIG. 2a is one of the first emission spectra of chemical interest, that of aniline at 30 deg C.; the upper spectrum shows transmittance spectrum of aniline, for comparison with lover trace that shows thermal emission spectrum.

FIG. 2b is: (A) absorption and thermal emission spectra of 220 mg/dL glucose in KBr sample at 41 deg C.; (B) thermal emission spectra from human plasma at 37 deg C. with a different glucose concentration. Peak intensities of deconvoluted spectral bands are shown in the insert.

FIG. 3 is a diagram of vibronic and radiative transitions for both in absorption (a)and in emission (b) of photons.

FIG. 4 is a simplified diagram of an embodiment of an instrument of the invention.

FIG. 5 is a simplified diagram of an other embodiment of an instrument of the invention: a) a remote sensor assembly inserted in ear canal; b) a analyzing electronics” microcomputer system and display.

### DETAILED DESCRIPTION

The present invention is directed at an instrument and a method for a noninvasive detection of the concentration of analytes in human body tissues, for example, glucose in blood, using naturally occurring infrared radiation in the micrometer spectral region of the human body heat emission. It relates more specifically to method and instrument that incorporates additional temperature and humidity sensors and allows better normalization of spectrally-specific analyte signal for a grater precision and accuracy of determination of the analytes' concentration and universal calibration in, for example, non-invasive blood glucose measurements in human subjects.

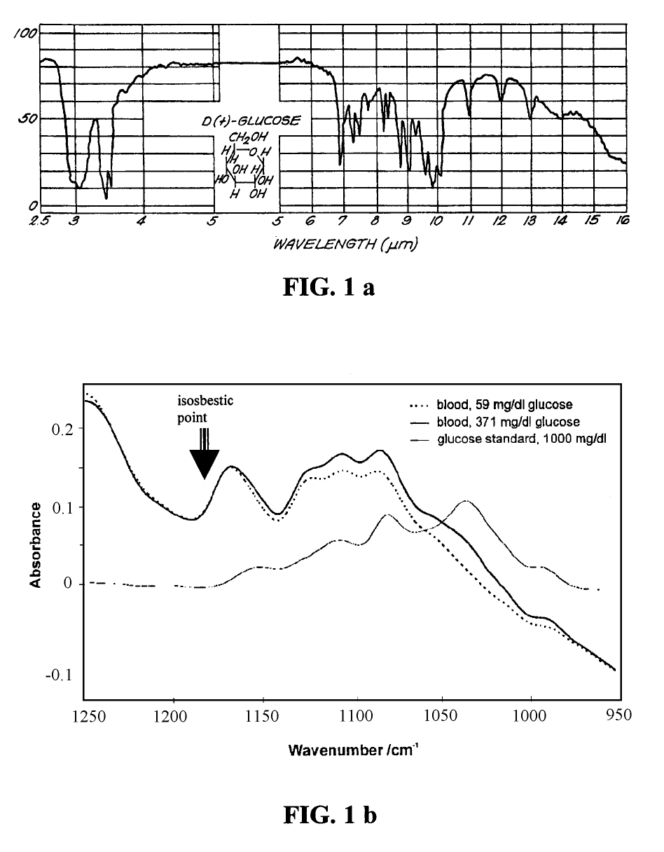

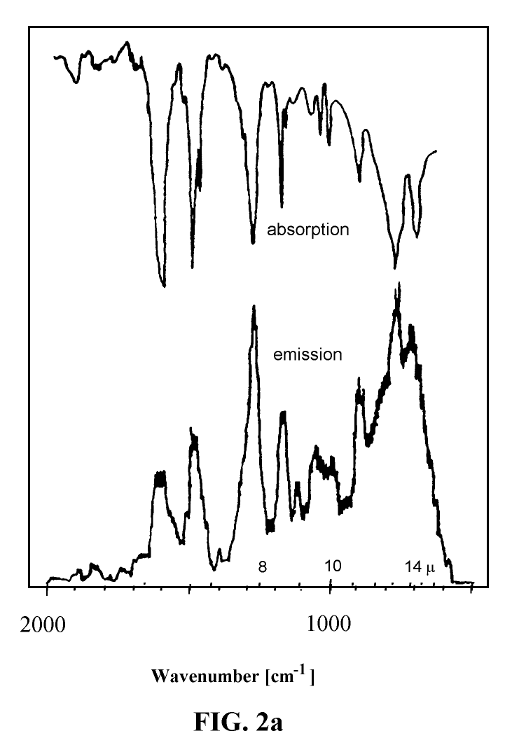

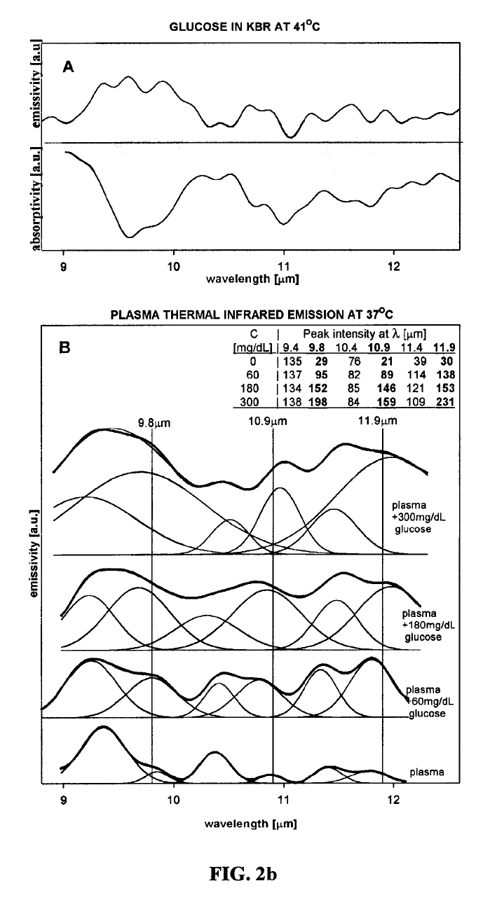

The scientific understanding that the molecular signature frequency of glucose is focused in the mid-infrared region, as shown on FIG. 1, and of the correspondence between the emission and absorption spectra, as shown on FIG. 2, have lead to the invention.

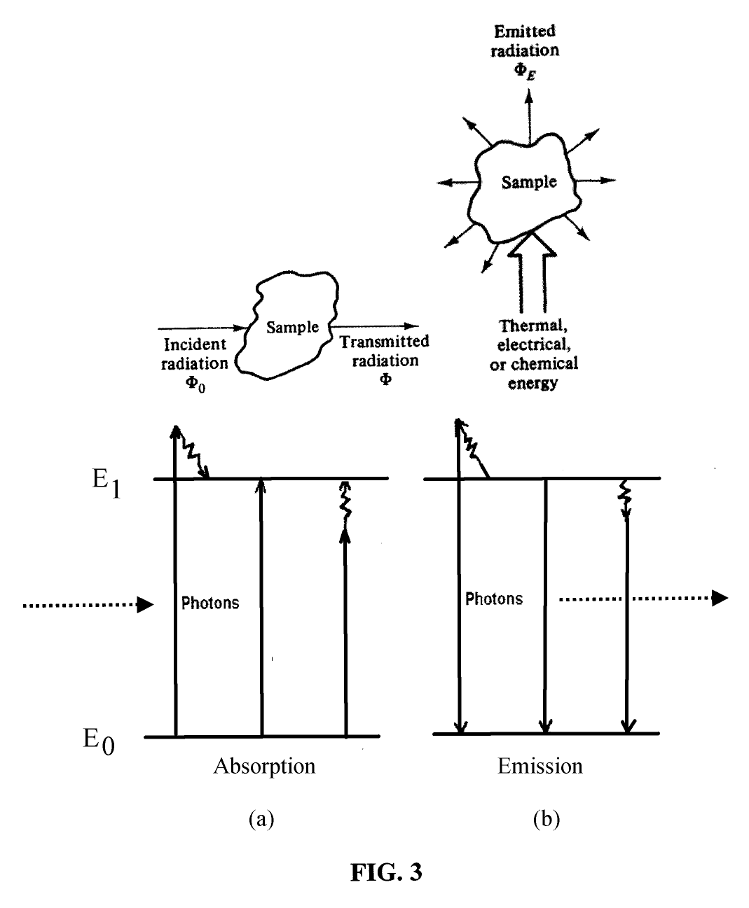

Absorption of radiation is characterized by selective removal or absorption of certain frequencies as radiation (incident radiation φ₀ passes through a substance (sample: solid, liquid, or gas) as shown on upper part of FIG. 3a. A molecule transition from one energy level (lower E₀) to another (higher E₁) occurs as shown on lover part of FIG. 3a. At room temperature most substances (molecules) are in the ground electronic state but can be thermally excited (radiation less) into higher vibrational energy levels and process of thermal emission (radiation) characterized by radiant flux φ_{E} will occur as shown on FIG. 3b. Emission of radiation is an inverse process of absorption. Relaxation of molecule to lower E₀ and more stable) energy state is accompanied by the release of a radiant photon (if selection rules allow it) of appropriate energy E₁ (frequency λ₁) as shown on lover part of FIG. 3b. For a thermally excited molecule, at a room or body temperature range, the thermal emission occurs in a mid-infrared wavelengths range.

Vibrational Spectroscopy could characterize molecules, which are composed of positive and negative ions that vibrate at quantized frequencies. When the positive and negative ions move out of phase with each other, absorption or emission of radiant energy becomes possible at the wavelengths corresponding to the vibrational frequency of the motions, as long as there is a net dipole moment. For example, in the glucose molecule the primary spectral absorptions or thermal emissions are due to the stretching motions modes of C—O and C—C and bending modes of O—C—H, C—C—H and C—O—H. The exact frequencies, shapes, intensities, and number of features in a spectrum are dependent on the relative masses, radii, distances, and angles between atoms and their bond strengths. These parameters are determined by the structural arrangement of the anions (i.e., their polymerization), and the location and composition of the cations associated with them. Because all molecules consist of unique structures and/or compositions, virtually every molecule has a different suite of vibrational absorption/emission characteristics and thus a unique spectrum in the thermal infrared radiation.

Glucose has a very well defined vibrational spectral feature in the fingerprint infrared region as shown, for examples, in FIG. 1a for infrared absorption spectrum of D-glucose and in FIG. 1b for infrared absorption spectra of blood added with glucose in the spectral range of the glucose absorption as well as on thermal emission spectra plots on FIG. 2b. The correspondence between the emission and absorption spectra was theoretically predicted by Planck (Planck Max, “The Theory of Heat Radiation”, New York, Dover Publications, 1991). Kirchhoff's law confirms that for the entire body in the same temperature and for the same wavelength, absorptivity is equal to monochromatic emissivity. Thus one can conclude that a tissue (e.g. blood) spectral characteristics with different contents of analyte (e.g. glucose) will show different emissivities of the tissue (e.g. tympanic membrane), which will make it possible to measure the concentration of an analyte (e.g. glucose) in the tissue (e.g. blood). Planck describes the difference between absorption spectroscopy and thermal emission phenomena where the entire volume of the emitting body is a source of radiation, which can be measured. One can observe the surface of a body as radiating heat to the surroundings but this does not imply that the surface actually emits heat radiation. The surface of a body never emits radiation but allows part of it coming from the interior to pass through. The other part is reflected inward. As the fraction transmitted is larger or smaller, the surface seems to emit more or less intense radiation. If one considers absorption, the external radiation could not be measured if the optical density of the sample is large (combination of thickness and absorption coefficient). In emission each and every infinitesimal internal part of a sample is a source of heat radiation. The surface allows the radiation to pass through from the interior and to be analyzed in the mid-infrared region.

One of the first thermal emission spectra (shown on FIG. 2a) of chemical interest that of aniline at 30 deg C. was shown experimentally in 1965 (from Griffiths P R, “Chemical IR Fourier Transform Spectroscopy”, New York, J. Wiley & Sons, 1975, FIG. 12.1, pp. 312) with its transmittance (absorption) spectrum for comparison. Infrared emission spectroscopy, while not commonly used, shows promise of application in several areas of chemical analysis. It was used, for example, during the Mars expedition (by NASA) to analyze the chemistry of Martian rocks and is used in astronomy to analyze chemical components of stars. For most of the samples measured there are excellent correspondences between band frequencies observed in the infrared emission spectrum and the absorption spectrum of the same material. For example, FIG. 2b shows absorption and thermal emission spectra of 220 mg/dL glucose in KBr pellet at 41 deg C., and the thermal emission spectra from human plasma at 37 deg C. with different glucose concentration. This figure emphasizes two important features: first is to show the spectral region of interest and second is to present experimental proof of the thermal emission detection ability of current room-temperature infrared detectors. Deconvolution shows bands sensitive and non-sensitive to glucose concentration changes in human plasma. For the viewing clarity, the spectra are upshifted along the vertical axis. The results of deconvolution in the inserted table show peak intensity changes versus glucose concentration. One can observe in the emission the corresponding bands of glucose absorption e.g. main band at 9.8 micrometers, band at 10.9 micrometers (corresponding to 914 cm-1 vibrational state of glucose) and a weaker band around 11.9 micrometers. The peak intensities of the deconvoluted spectral bands that are shown in the figure insert, part B of FIG. 2b, follow the glucose concentration changes.

The invented method and instrument is an improvement of a prior art analytical means of Thermal Emission Spectroscopy (TES) to measure infrared radiation emitted naturally by the human body. This infrared radiation contains spectral information of the emitting body tissue. The radiation thermometer measures the integral energy of infrared radiation from the body, through the entire infrared wavelengths and without spectral discrimination. In the case of the prior art instrument the signal from the detector is proportional to the difference between the intensity of the spectrum emitted from the body passing through the filter with the spectral characteristic of the measured analyte, for example, glucose in blood, and the intensity of the infrared spectrum emitted from the body passing through the filter with spectral characteristics which do not include spectral bands of the analyte. If signal passing trough both filters is well balanced then the measured signal should be independent from the overall temperature of the emitting body because this information is canceled out by subtraction. The same applies to other spectral intensity changes that are independent of the analyte concentration changes.

It was discovered that the above assumptions are valid only for well-balanced intensities passing through both filters of the infrared detector. In addition, the infrared detector signal's dependence on the ambient and the detector base temperature has influenced the resulting differential signal. Other parameters that have influence on the detector glucose spectral signal were the size and shape of the ear canal (e.g. the detector's distance from the tympanic membrane) and the ambient humidity. The invented improved method and the improved instrument is directed to minimize the effects of these parameters on spectral analyte signal of thermal emission from the tissue.

The prior art method and instrument follow up independent clinical studies are described in the publication “A NOVEL NONINVASIVE BLOOD GLUCOSE MONITOR” by Malchoff et al., Diabetes Care, 25(12) 2268-2275, 2002, which are also hereby incorporated herein as reference. In this study two-window infrared non-invasive blood glucose ear monitor was used, according to the teaching of U.S. Pat. No. 5,666,956. The subject's oral and ear temperature, room temperature, and room humidity were recorded during the study. Measurements of infrared ear temperature were made after each spectral thermal infrared emission glucose measurement. Environmental parameters as well as body physiological parameters were measured using outside instruments. Ambient temperature and humidity was measured using Radio Shack Digital Thermo-Hygro (Cat. No. 63-1013) thermometer and humidity gauge. Subjects” oral temperature was measured using mercury thermometer and their ear tympanic temperature was measured using OMRON MC-505 infrared thermometer.

Ear temperature together with body temperature measured by conduction has an important role in the ability to achieve universal calibration. For different lengths of the ear canal e.g. different distance between detector and tympanic membrane, these measurements normalize the variability of such differences in the use of the device. Intensity of infrared heat radiation measured by the detector is defined by temperature of the radiating body according to Planck's/Kirchoff's laws, its emissivity and a distance between detector and emitting tissue, e.g. tympanic membrane. Due to various shape and size of ear canal in various subjects, the distance between detector and tympanic membrane vary. By introducing ear temperature of the subject with his body temperature as normalization factors, the normalized signals become independent of these physiological differences. Previously both measurements were made using two separate instruments, an electronic ear thermometer and an oral mercury thermometer. Errors of these measurements that were not performed at the same time and were not performed out of the same spot on the tissue contributed to increased errors in resulting concentration values. Both of these temperature measurements are integrated into the novel and improved invented instrument. The replacement of the independent electronic ear thermometer will be accomplished in a two-window design by using a differential amplifier connected to two (reversibly polarized) detector sensors, for example thermopile, designed to generate two output signals. One will be a differential signal of the glucose signature; the other signal will be the background, so called quasi-isosbestic point of the spectrum, of the intensity not changing with the analyte concentration changes and proportional to the ear temperature. In a four-window detector design, ear temperature measurements will be accomplished by measuring the infrared radiation over a wide range of energy from 8 to 14 microns in one of the four windows. The remaining windows will incorporate infrared filters for the glucose signature and quasi-isosbestic point of the spectrum.

The quasi-isosbestic point (one of possible isosbestic point is indicated approximately on FIG. 1b) in the emission/absorption mid-infrared spectrum of glucose solution was derived from well-controlled spectral studies of various glucose concentrations in water (ATR) solution and in blood and blood plasma (absorption). Intensity (of mid infrared absorption or, by correspondence, thermal emission) at this isosbestic point of spectrum is not changing with the changes of glucose concentration. In the real world the intensity of the measured spectrum is influenced by many factors such as for example: sample (tissue, blood, body) temperature, sample water contents (glucose spectral signature is on top of very broad water spectrum), sample other constituencies (other chemicals, interfering substances), and also by the device detection system emissivity, efficiency and throughput. This is why one needs some normalization point to be able to compare different spectra if needed information's relates to the spectral line intensities. In the invented novel device the spectrum isosbestic point is used as a reference in differential detection system (double windows filtometer) to normalize and reduce influence of the above-described factors. The difference between the laboratory situation for spectrum normalization and the real world condition depends on many additional factors. If, for example, the distance between the detector device and the tympanic membrane will be constant in every measurement case, if the relation between the thermal emission intensity at isosbestic point and the intensity at glucose signature wavelength with target (tissue) temperature will be known and well defined, if the relation between emissivity of optical system including thermopile detector will be known and well defined, etc., it will be not necessary to measure all outside parameters to achieve universal calibration. The isosbestic point compensates only for a part of the factors that are responsible for the difference between well-controlled laboratory conditions (laboratory measured thermal emission spectra on FIG. 2b) and a real world situation (different subjects with wide range of subject conditions, e.g. shape of ear canal in wide range of ambient conditions).

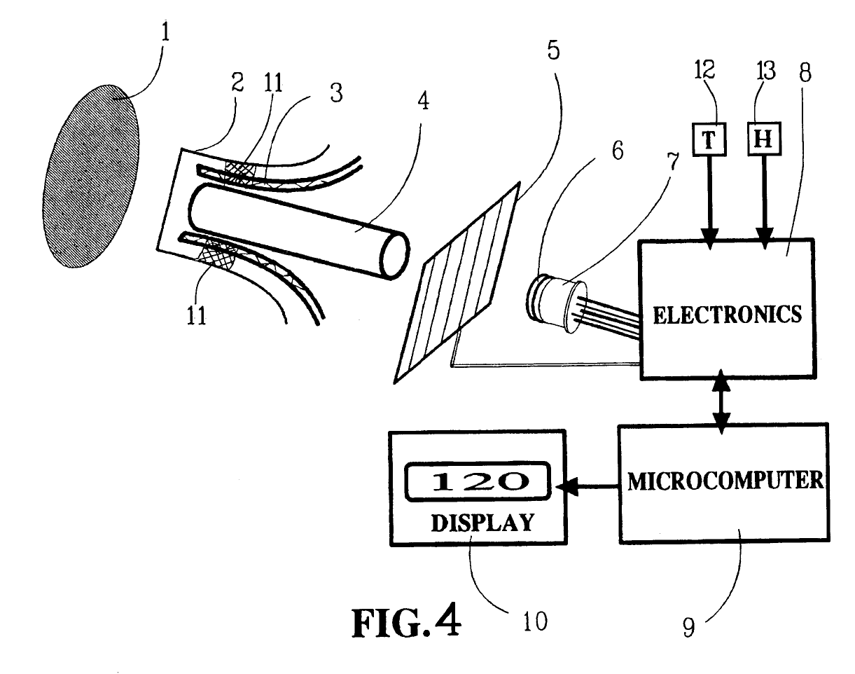

The FIG. 4 shows a simplified diagram of an embodiment of the invented instrument. Infrared radiation from the object target 1 such as a human body, or for example its tympanic membrane, is optically received by invented instrument. The instrument consists of: the speculum 3 (for insertion, for example, into an ear canal) with an optional plastic cover 2 (for hygienic reasons, fabricated of a thin polymer material that is transparent to radiation in the far infrared spectral region); the infrared optical system which can include: the infrared wave guide 4 such as a hollow tube polished and/or gold plated inside, or in other form being selected from the group consisting of a mirror, reflector, lens, and a fiber optics transmitting infrared radiation made, for example, from ATRIR special glass produced by Amorphous Materials, Inc.; the optional optical valve 5; and the detecting system with electronics 8, microcomputer 9, a display system 10, body temperature sensor 11 and sensors for ambient temperature 12 and optionally for humidity 13. The said infrared wave-guide 4 can be in the form of any directing device such as a mirror, reflector, lens, etc. On the end of the receiving waveguide 4 an optional optical valve 5 could be positioned in the form of a shutter or chopper that optionally activates measurements of infrared radiation by a detecting system. The detecting system consists of an optical infrared filter set 6 and a detector 7 sensitive in the infrared region of human body radiation. This infrared sensor (detector 7) can be of any type known to the art. This sensor generates an electrical signal, which is representative of the received radiation and includes a signal of the infrared specific analyte emission and a signal related to body infrared temperature emission. The other detector 7 signal received by the conditioner electronics 8 is signal from the detector's base thermistor (not shown) required for normalization of detector 7other infrared radiation signals. The electronics 8, microprocessor 9 and the display system 10 have to stabilize the temperature dependent parts of the instrument, compensate for the ambient and body temperature changes, compensate for ambient humidity changes, then correlate, calculate and further display the concentration of the analyte from the spectral intensity measurements of the infrared radiation emitted by the body.

The detection system comprises an infrared energy sensor 7for infrared energy measurements and could consist, for example, of the dual element pyroelectric or thermopile detector or any other infrared energy detector known in the art. Infrared energy sensor could comprise for example two sensing areas covered by a silicon window (optical infrared filter set 6) with a long pass filter to pass only infrared radiation, which corresponds to emission in the range of the internal temperature of a human body. Said infrared sensor could comprise more sensing areas such as three, four, etc. Any combination of infrared filters 6 could cover the sensing elements. In case of infrared sensor with two sensing areas, the spectrally modified infrared radiation from, for example, the tympanic membrane illuminates both windows (sensing areas), one with a negative correlating filter which blocks radiation in the absorption bands for the analyte to be measured and the other which passes through a neutral density filter capable of blocking radiation equally at all wavelengths in the range of interest. It is to compensate for overall attenuation by the negative correlating filter in the first sensing area. The two sensing areas are connected so that their outputs are subtracted. Difference of the radiation intensity between the two radiation paths provides a measure proportional to the analyte concentration. The electrical signal from the infrared detector, including also the body infrared temperature, is then sent to the forming electronics 8 system. Signal from body temperature sensor 11, ambient temperature sensor 12 and ambient humidity sensor 13 is also input into the forming electronics. Then all signals are further sent to the microcomputer 9 and to the display 10 system as shown in FIG. 4. Any combination of interconnections of sensors with forming electronics and microcomputer, needed to achieve the intended result, can be used. Microcomputer 9 has the role to correlate, calculate and further display the concentration of the analyte resulting from the spectral intensity measurements of the infrared radiation emitted by the body.

One can also use a narrow band filter with the spectral characteristic specific to the analyte infrared signature in front of one of the windows (sensing elements) and cover the other by an appropriate attenuation filter or another narrow band filter with a spectral characteristic at a wave-length not sensitive to the analyte concentration (for example at isosbestic point). Careful adjustment of the peak wavelength and transmission of both narrow band filters can compensate for changes in body temperature but is not necessary if other compensation means for normalization are to be used. In a multi-window system, one can use, for example, one of the sensing elements for body infrared temperature measurements in, for example, the 8 to 14 micrometer spectral range of infrared spectrum.

An infrared wave-guide 4, as a part of the optical detection system, must scramble and direct infrared radiation from the tympanic membrane to the detector windows. The one possible design could be the wave-guide made of an inner-diameter gold-plated, polished tube attached mechanically to the detector housing. The diameter of the tube must be sufficiently large to illuminate equally all detector windows. It also must be sufficiently small to be accommodated into the speculum designed for insertion into ear canal (diameter of about 5-6 mm). The scrambling of the radiation in order to discard its directional properties is achieved by choosing the optimized length-to-diameter ratio of the tube. A design of the assembly of the detector and the infrared wave-guide has to fit different diameters of the speculum required for both adult and younger pediatric use. Separate modules could to be used to accommodate different size ranges of ear canals. Said infrared wave-guide could be also selected among other optical elements such as a mirror, reflector, lens, and a fiber optic. The directional properties of the infrared wave-guide and incorporation of infrared temperature sensor into the infrared emission analyte detector system will assure that both the spectral analyte specific emission intensities and the ear temperature are measured at the same spot of the emitting tissue e.g. tympanic membrane. In prior art measurements performed by two instruments, e.g. non-invasive glucose monitor and ear thermometer, various spot of measurements contributed to increased uncertainty of resulting analyte concentration.

In order to stabilize and normalize for environmental and subject variability, the invented instrument will include sensors for ambient temperature 12, humidity 13 and for measurement of the subject's body temperature 11 by conduction. In the prior art instrument, there were no “built in” sensors for ambient temperature, humidity and body temperature. The temperature measurements are important for compensation for changes in the thermal emission spectra that are not related to analyte concentration changes. The optional humidity measurements are important for compensation for the possible interference of water vapor in the measured infrared spectral radiation range. Water vapor could influence, like a neutral density filter, the overall spectral infrared signal intensity in the region of glucose spectral signature. Water spectrum in the glucose infrared signature region is not specific but its changes due to its concentration variation in air (humidity) could influence glucose signature baseline. With a higher humidity the detected infrared spectral signal is weaker. A wide selection of commercially available sensors could be used. The mechanical design incorporates the sensors into the monitor housing and speculum 3 or optional speculum plastic cover 2. Electronics 8 and signal conditioners will support the sensors requirements.

Temperature sensing chips such as AD592 by Analog Devices as well as standard thermistors for ambient temperature measurements could be used. Humidity sensors such as HIH-3602 Monolithic Integrated Circuits could be used. Body temperature by conduction could be measured using a temperature sensor known in the art. It could be achieved, for example, by thermistor(s) incorporated into the speculum 3 or into the optional speculum plastic cover 2, with a resistant (e.g. platinum) wire placed around the distal part of the speculum 3 or the optional speculum plastic cover 2 or using appropriate heat flux sensors placed on speculum 3 or on its plastic cover 2. The thermal mass of the temperature sensor, for example, the thermistor, resistant wire, heat flux sensors and appropriate construction materials of speculum 3 and optional speculum plastic cover 2 will have to satisfy requirements for rapid thermal conduction since it is preferable for reproducible temperature measurement to be completed within a short time period (e.g. about 6 to 10 seconds) of speculum 3 insertion into the ear canal.

The molded speculum 3 of invented instrument with imbedded temperature sensor 11, covered by an optional plastic cover 2 made of material transparent to radiation in an infrared spectral region, with the optional embedded temperature sensor 11, is inserted in the ear canal. Sensors output signals such as the infrared emission differential signals of analyte signature, the body internal temperature, the ear temperature and humidity sensor output signals make the completed necessary information to achieve universal calibration. All the signals in a form of electrical signal are then input into the conditioning electronics 8, and finally into the microcomputer 9 for signal evaluation. Results of the signal evaluation are then displayed on the instrument display 10 as the concentration of the measured analyte.

The present invention is still directed at an improved instrument and improved method for the continuous non-invasive detection of the concentration of analytes in human body tissues, for example, glucose in blood, using naturally occurring infrared radiation in the micrometer spectral region of the human body. The invented instrument will measure continuously infrared radiation emitted naturally by the human body and normalize the measured signal using signals from variety of temperature and optionally humidity sensors for analyte concentration determination in continuous manner.

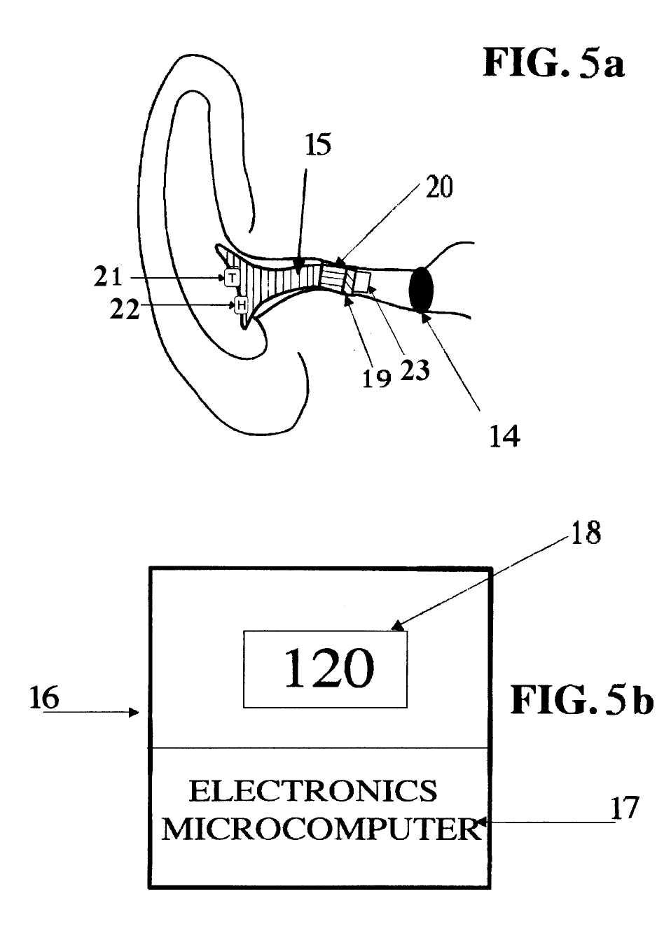

FIG. 5a and FIG. 5b shows a simplified diagram of a further embodiment of the invented instrument. A remote sensor assembly inserted in a subject's ear canal optically receives infrared radiation from the object target 14 such as a human body tympanic membrane. The infrared radiation sensor is contained within an earplug remote assembly 15, which is connected with an electronic analyzing unit 16 by cable or by a telemetric transmitting and/or receiving system. The instrument consists of the earplug 15 (for insertion into the ear canal) with the infrared radiation sensor detecting system, and the electronic analyzing unit 16 consisting of: electronics with the microcomputer 17 and the display system 18. The earplug assembly 15 optionally would consist of the telemetric transmitting electronics while the electronic analyzing unit 16 optionally would consist of the telemetric receiving electronics. The infrared radiation sensor detecting system consists of an optional infrared wave-guide 23, of the optical infrared filter set 19 and the infrared detector 20 sensitive in the infrared region of human body radiation. This infrared radiation sensor (detector 20) can be of any type known to the art which allows continues measurement of infrared energy, including a thermopile sensor. This sensor generates an electrical output signal that is representative of the received infrared radiation. It includes the signal of the infrared specific analyte emission and the signal related to the body infrared temperature emission. Still another detector 20 signal received by forming electronics and microcomputer 17 is the signal from the detector base thermistor (not shown) required for normalization of various infrared radiation signals of detector 20. The earplug 15 consists of the ambient temperature sensor 21 and, optionally, the ambient humidity sensor 22. The electronics with microprocessor 17 and the display system 18 must stabilize the temperature dependent parts of the instrument, compensate for the ambient temperature changes detected by ambient temperature sensor 21, optionally compensate for the ambient humidity changes detected by ambient humidity sensor 22, and then correlate, calculate and further display the concentration of the analyte from the spectral intensity measurements of the infrared radiation emitted by the body. The electronics with micro-processor 17 can be optionally connected directly to a regulated insulin reservoir such as an insulin pump or an artificial pancreas for an automatic insulin control system.

The present invention reduces variability of the spectral signal of the analyte of interest due to environmental conditions such as ambient temperature and humidity, physiological body conditions such as body temperature, infrared radiation measured tissue temperature, the varying distance between spectral detector and emitting tissue, the varying spot of the tissue non-contact temperature measurements in comparison to the spot where the spectral data are collected. It is aimed at reducing the number of variables of data analysis by mathematical methods. The mathematical methods of analysis include partial least squares, principal component analysis, artificial neural networks, mixture of expert's algorithm, chemometric techniques, mathematical models, and the like.

The present invention provides optimal means for measurement of the concentration of the analyte of interest from the infrared energy emissions of the tissue by means of evaluation of the temperature and humidity parameters influence on analyte concentration derived from infrared spectral emission signal. The method and instrument uses the steps of sensing the infrared thermal emission analyte signal level, sensing the body and ambient temperature, sensing the ambient humidity, producing output electrical signals representative to the said physical quantities, converting the resulting input, and sending the converted input to a processor. The microcomputer is adapted to provide the necessary analysis of the signal to determine concentration of the substance of interest and display the concentration of the substance of interest.

The invention is aimed to monitor ambient conditions and multiple physiological variables of a patient at a single site, using multiple sensors integrated into a single instrument. The instrument has the infrared spectral sensor, the detector base temperature sensor, the infrared temperature sensor and the body temperature conduction sensor, the ambient temperature sensor, the humidity sensor and a communication circuit for outputting the information produced by said sensors. These elements are integrally placed within the housing of the instrument or within the speculum 3, or within optional speculum plastic cover 2 or within the earplug 15 mold made to fit the ear of the patient.

The embodiments of the present invention are intended to be merely exemplary and those skilled in the art shall be able to make numerous variations and modifications to it without departing from the spirit of the present invention. All such variations and modifications are intended to be within the scope of the present invention as defined in the appended claims.

1. An improved method of determining a human body tissue analyte concentration by non-invasive measurement of emission spectral lines characteristic to a body tissue analyte in an infrared spectral region emitted naturally by a human body as heat, comprising:
a) measuring a spectral intensity of said emission lines;
b) said emission spectral lines having a wavelength dependence of tissue constituents;
c) detecting the emission spectral lines at a predetermined emission wavelength;
d) analyzing the emission spectral lines in said infrared spectral region;
e) measuring ambient temperature;
f) measuring optionally ambient humidity;
g) measuring body temperature by means of heat conduction;
h) measuring body temperature in a non-contact manner by means of radiation;
i) correlating said spectral intensity of emission spectral lines, said ambient temperature and said optional humidity, said body temperature measured by means of conduction and means of radiation with body analyte concentrations.

2. The improved method as in claim 1, for determining blood glucose concentration by non-invasive measurements of emission spectral lines characteristic to a body tissue analyte in an infrared spectral region emitted naturally by a human body's tympanic membrane in an infrared wavelength spectrum as heat including measuring of ambient and body temperature and optionally ambient humidity.

3. An improved instrument for determining a human body tissue analyte concentration by non-invasive measurement of emission spectral lines characteristic to a body tissue analyte in an infrared spectral region emitted naturally by a human body as heat, comprising:
a) a means for detecting said emission spectral lines at a predetermined infrared wavelength;
b) a means for detecting a spectral intensity of the emission spectral lines;
c) a means for measuring ambient temperature;
d) an optional means for measuring ambient humidity;
e) a means for measuring body temperature by means of heat conduction;
f) a means for measuring body temperature in noncontact manner by means of radiation;
g) a means for correlating said spectral intensity of emission spectral lines, said ambient temperature and said optional humidity, said body temperature measured by means of conduction and means of radiation with body analyte concentrations.

4. The improved instrument of claim 3 wherein the detecting means comprises:
a) a detector means; and,
b) an analyzing means in the form of a wavelength selecting means for the emission spectral lines;
said detector means comprising means for detecting the intensity of received emission spectral lines from said analyzing means producing an electrical output signal;
said wavelength selecting means comprising means for allowing only significant wavelengths of tissue analyte emission spectral lines in natural infrared radiation emitted by the human body to reach the detector means.

5. The improved instrument of claim 3 wherein the measuring means comprises sensors for said temperature and optionally sensors for said humidity measurements.

6. The improved instrument of claim 4, wherein the detector means comprises an infrared energy sensor for infrared energy measurements.

7. The improved instrument of claim 4, wherein the analyzing means comprises filter means for filtering the emission spectral lines to allow only for wavelengths significant to the tissue analyte emission spectral lines to pass or to be absorbed before reaching the detector means.

8. The improved instrument of claim 3, where the correlating means is an electronic means comprising electronics and a microcomputer for correlating the electronic output signal from the detecting means and measuring means with the tissue analyte concentration.

9. The improved instrument as in claim 3, for determining blood glucose concentration by non-invasive measurements of emission spectral lines characteristic to blood glucose as a body tissue analyte.

10. An improved instrument for non-invasive tissue analyte concentration measurements based on measurements of emission spectral lines characteristic to a human body tissue analyte in an infrared spectral region emitted naturally by a tympanic membrane as heat, comprising:
a) an ear plug assembly for insertion into an ear canal;
b) said ear plug assembly comprising an infrared radiation detecting system comprising an optical infrared filter set and a detector sensitive in an infrared region of human body heat radiation for detecting the emission spectral lines, and providing an output based thereon;
c) said ear plug assembly comprising a body temperature measurements sensor by means of conduction;
d) said ear plug assembly comprising a body temperature measurements sensor by non-contact manner by means of radiation;
e) a sensor for ambient temperature measurements;
f) an optional sensor for ambient humidity measurements;
g) said ear plug assembly and said sensors comprising connection means whereby the output of the detecting system may be connected with electronics, a microcomputer and a display system for forming, calculating, and displaying an electrical signal from the said detecting system and said sensors to show a numerical value of the analyte concentration.

11. The improved instrument of claim 3 and 10 wherein said detecting system incorporating body temperature sensor is adapted to be in thermal conductive contact with a human body.

12. The improved instrument as in any one of claims 3, 4, 5, 6, 7, 8, 9, 10 or 11 wherein said detecting of said emission spectral lines and said spectral intensity of the emission spectral lines and said detecting of temperature and optionally humidity are effected continuously.

13. The improved instrument of claim 12 wherein the emission spectral lines are the emission spectral lines of blood glucose.

14. The improved method as in any one of claims 1 or 2 wherein the measuring of the spectral intensity of said emission lines and the detecting of the emission spectral lines and said detecting of temperature and optionally humidity are effected continuously.

15. The improved method as in claim 14 wherein the emission spectral lines are the emission spectral lines of blood glucose.

16. An improved instrument for determining a human body tissue analyte concentration by non-invasive measurement of emission spectral lines characteristic to a body tissue analyte in an infrared spectral region emitted naturally by a human body as heat, comprising:
a) a speculum for insertion into an ear canal;
b) an optional plastic cover made of material transparent to radiation in an, infrared spectral region;
c) an infrared wave-guide for receiving infrared radiation from the tympanic membrane and for illuminating all windows of a detecting system;
d) said infrared wave-guide being selected from the group consisting of a mirror, reflector, lens, hollow tube, and a fiber optic;
e) the detecting system consisting of:
i) an infrared filter set; and,
ii) a detector sensitive in an infrared region of human body heat radiation;
f) an optical infrared filter set consisting of a negative correlating filter or narrow band filters;
g) a detector system sensitive in an infrared region of human body heat radiation consisting of at least two sensing areas electronically connected so that their outputs are subtracted;
h) the detector system comprising a body temperature sensor by non-contact means e.g. radiation;
i) said speculum optionally comprising a body temperature sensors by conduction;
j) a sensor for ambient temperature measurements;
k) an optional sensor for ambient humidity measurements; and,
l) said detector and said sensors having an output connected with electronics, a microprocessor and a display system for forming, calculating, and displaying a resulting electrical signal from the detector and sensors to show a numerical value of the analyte concentration.

17. An improved instrument for non-invasive tissue analyte concentration measurement based on measurement of emission spectral lines characteristic to human body tissue analyte in an infrared spectral region emitted naturally by tympanic membrane as heat, comprising:
a) a speculum for insertion into an ear canal and for receiving from an infrared wave-guide infrared radiation from the tympanic membrane and for illuminating all windows of a detecting system;
b) the detecting system comprising:
i) an optical infrared filter set consisting of a negative correlating filter or narrow band filters; and,
ii) a detector sensitive in an infrared region of human body heat radiation, said detecting systems positioned to be illuminated by infrared radiation arriving from said optical infrared filter set, or negative band filters, and having at least two sensing areas electronically connected so that their outputs are subtracted to produce a detection output;
iii) a body temperature sensor by non-contact means e.g. radiation;
c) said speculum optionally comprising a body temperature sensors by conduction;
d) a sensor for ambient temperature measurements;
e) an optional sensor for ambient humidity measurements; and,
f) said detector and said sensors having an output connected with electronics, a microprocessor and a display system for forming, calculating, and displaying an electrical signal from the detector and sensors to show a numerical value of the analyte concentration.

18. An improved instrument as in claim 17 wherein the infrared wave-guide is selected from the group consisting of a mirror, reflector, lens, hollow tube, and a fiber optic.

19. The instrument as in any one of claims 17 or 18 wherein the emission spectral lines are the emission spectral lines of blood glucose.
